(19) United States
(12) Patent Application Publication
(10) Pub. No.: US 2025/0177738 A1
(43) Pub. Date: Jun. 5, 2025

(54) IMPLANTABLE APPARATUS HAVING MARKERS FOR DETERMINING PENETRATION DEPTH

(71) Applicant: MEDTRONIC, INC., Minneapolis, MN (US)

(72) Inventors: Hongyang Lu, Shanghai (CN); Xue Zhang, Shanghai (CN); Zheng Wang, Shanghai (CN); Juan Meng, Shanghai (CN)

(21) Appl. No.: 18/845,937

(22) PCT Filed: Mar. 17, 2022
(86) PCT No.: PCT/CN2022/081370

§ 371 Date: Sep. 11, 2024

(51) Int. Cl. A61N 1/05; A61B 90/00

(52) U.S. Cl. CPC A61N 1/0587; A61B 90/39; A61B 2090/3966; A61B 2090/3983

(57) ABSTRACT

An implantable apparatus (100) and methods thereof. The implantable apparatus (100) includes a body (110) defining a distal end region (112) extending along a distal end region axis (111). The body (110) is configured to be inserted into cardiac tissue of a patient's heart at a target site. The implantable apparatus (100) also includes a plurality of markers (120) located along at least a portion of an outer surface of the distal end region (112) of the body (110). The plurality of markers (120) define a first configuration when located within the cardiac tissue and a second configuration when not located within the cardiac tissue. The first configuration is different than the second configuration.

This disclosure generally relates to medical devices, such as implantable stimulation leads, including a plurality of markers.

Medical catheters and leads are commonly used to access vascular and other locations within a body and to perform various functions at those locations, for example, delivery catheters may be used to deliver medical devices, such as implantable medical leads.

Alignment of features of a medical device in the body can be critical when deploying treatment to specific locations within the anatomy. Three-dimensional spatial orientation when navigating, delivering, and/or implanting an implantable apparatus (e.g., lead, catheter, or other implantable device) can be difficult while looking at imaging. For example, it is known that some implanters may believe that the implantable device (e.g., lead) they are implanting is located proximate the septum of the heart when, in reality, it is located proximate the free wall of the heart.

Specifically, medical pacing leads may be fixed (e.g., screwed) deep into the interventricular septum to capture the left bundle branch. The depth of the medical lead may be important because the left bundle branch may not be captured if the lead tip of the medical lead does not go deep enough to reach the left bundle branch. Additionally, perforation may occur if the lead tip goes too deep. In other words, the precise depth of the lead tip of the medical lead may be important.

Often, the medical lead may be visible under x-ray, however, the myocardium (e.g., septum) may be invisible. As such, contrast injection may be utilized in an attempt to check the depth of the lead. However, the contrast may be difficult to interpret for various reasons including, for example, heart beating, angle of view, blurry boundary, contrast flow, etc.

### SUMMARY

The techniques of the present disclosure generally relate to structures and methods to assist in indicating a depth of a medical device (e.g., a pacing lead) in cardiac tissue. Specifically, the present disclosure utilizes markers (e.g., materials that may be visible under imaging) as physical structures on the surface of the medical device (e.g., near a distal end region) that can be used to determine depth of penetration. For example, the markers may be spaced apart by a fixed and known distance (e.g., a preset distance) such that the relationship between a distal end (or features) of the medical device and the markers is known and can be used to determine the depth of the medical device (or features thereon) within tissue (e.g., at nearly any angle of view).

For example, the markers may be positioned near the distal end region of the medical device such that only a portion of the markers are inserted into the tissue (e.g., inserted with the medical device), while the remainder of the markers remain outside of the tissue. All of the markers may have the same appearance or configuration prior to being inserted into tissue. After at least a portion of the medical device is inserted into the tissue, the markers that are located within the tissue may be configured differently than the markers that are not located within the tissue. For example, the markers located within the tissue may have a different visual appearance (e.g., under imaging) than the markers not located within the tissue. In other words, the markers may change appearance or configuration when interacting with or being inserted into the tissue. Therefore, the markers may be evaluated (e.g., counting the markers having a different appearance or configuration) to determine the depth of the medical device within the tissue.

The markers may include a variety of different structures and/or materials. For example, in one or more embodiments, the markers may include threadlike, soft, and slippery (e.g., like hair) structures (e.g., filaments) extending from the medical device that are fixed in rows with a fixed (e.g., preset) distance between each marker to show the depth of the medical device penetration. For example, the threadlike structures not positioned within the tissue may freely extend away from the medical device, while the threadlike structures positioned within the tissue may bend and contact the medical device (e.g., matted down). As such, the number of unaffected threadlike structures may be counted, while viewing under imaging, to determine the number of threadlike structures within the tissue. The number of threadlike structures then relates to the depth of the medical device within the tissue (e.g., because of the known fixed positioning of the threadlike structures).

One illustrative implantable apparatus may include a body defining a distal end region extending along a distal end region axis and a plurality of markers. The body may be configured to be inserted into cardiac tissue of a patient's heart at a target site. The plurality of markers may be located along at least a portion of an outer surface of the distal end region of the body. The plurality of markers may define a first configuration when located within the cardiac tissue and a second configuration when not located within the cardiac tissue. The first configuration may be different than the second configuration.

One illustrative method may include positioning a body of an implantable apparatus proximate a target site comprising cardiac tissue of a patient's heart. The body may define a distal end region extending along a distal end region axis. The implantable apparatus may also include a plurality of markers located along at least a portion of an outer surface of the distal end region of the body. The method may also include inserting at least a portion of the distal end region of the body into the cardiac tissue of the patient's heart at the target site such that a first portion of the plurality of markers may be located within the cardiac tissue and a second portion of the plurality of markers may be located outside of the cardiac tissue. The first portion of the plurality of markers may be configured differently than the second portion of the plurality of markers when the first portion is within the cardiac tissue. Further, the method may include determining a depth of the body within the cardiac tissue based on a length of the first portion of the plurality of markers.

Another illustrative implantable apparatus may include a body, one or more markers, and a jacket. The body may define a distal end region extending along a distal end region axis. The body may be configured to be inserted into cardiac tissue of a patient's heart at a target site. The one or more markers located along at least a portion of an outer surface of the distal end region of the body. The jacket may be configured to move relative to the body. The jacket may be restricted from being inserted into the cardiac tissue such that the jacket moves relative to the body when the body is inserted into the cardiac tissue. The jacket may include reference markings configured to be compared to the one or more markers of the body to determine a depth of the body within the cardiac tissue.

Another illustrative method may include positioning a body of an implantable apparatus proximate a target site comprising cardiac tissue of a patient's heart. The body may define a distal end region extending along a distal end region axis. The implantable apparatus may also include one or more markers located along at least a portion of an outer surface of the distal end region of the body. The method may also include positioning a jacket over the body. The jacket may include reference markings. Further, the method may include inserting at least a portion of the distal end region of the body into the cardiac tissue of the patient's heart at the target site. The jacket may be restricted from being inserted into the cardiac tissue such that the jacket moves relative to the body. The method may also include comparing the reference markings of the jacket to the one or more markers of the body to determine a depth of the body within the cardiac tissue.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is an illustrative view of an implantable apparatus including a plurality of markers and implanted in a septal wall of the right ventricle according to the present disclosure.

FIG. 2A is a conceptual diagram of an illustrative implantable apparatus having filaments according to the present disclosure.

FIG. 2B is an axial view of the conceptual diagram of the implantable apparatus of FIG. 2A.

FIG. 3 is a conceptual diagram of the implantable apparatus of FIG. 2A positioned relative to a septal wall.

FIG. 4 is a conceptual diagram of another illustrative implantable apparatus having balloons according to the present disclosure.

FIG. 5 is a conceptual diagram of the implantable apparatus of FIG. 4 positioned relative to a septal wall.

FIG. 6 is a conceptual diagram of yet another illustrative implantable apparatus having a material coating according to the present disclosure.

FIG. 7 is a conceptual diagram of the implantable apparatus of FIG. 6 positioned relative to a septal wall.

FIG. 8 is a conceptual diagram of an illustrative implantable apparatus having a jacket that is movable relative to a body portion of the implantable apparatus according to the present disclosure.

FIG. 9 is a conceptual diagram of the implantable apparatus of FIG. 8 positioned relative to a septal wall.

FIG. 10 is a flow diagram that illustrates one example of a method for determining a depth of a body of an implantable apparatus within cardiac tissue.

FIG. 11 is a flow diagram that illustrates another example of a method for determining a depth of a body of an implantable apparatus within cardiac tissue.

### DETAILED DESCRIPTION

The present disclosure generally describes systems and methods for determining a depth of a medical device within tissue (e.g., cardiac tissue of a patient's heart at a target site) when viewed under imaging. For example, the medical device may include a variety of different physical structures or markings located at a distal end region of the medical device to assist in determining the depth of the medical device within the tissue. Specifically, the physical structures or markings may include filaments, balloons, material coatings, etc. The physical structures or markings may define a configuration or appearance when not positioned within the tissue and may be modified or changed to another configuration or appearance when positioned within the tissue (e.g., due to interactions with the tissue). Therefore, because the locations of the physical structures or markings are fixed and the distances between the markings and medical device are known, the depth of the medical device within the tissue can be determined based on evaluating the locations of the different configurations.

Further, in one or more embodiments of the present disclosure, the system and methods may also include a jacket that moves relative to the medical device to assist in determining the depth of the medical device within the tissue when viewed under imaging. The medical device may include markers disposed on an outer surface of the medical device and the jacket may include reference markings. The medical device may be inserted into the tissue and the jacket may be restricted from being inserted into the tissue. Therefore, the jacket may move relative to the medical device and, thus, the reference markings on the jacket may move relative to the markers on the medical device. Because the starting point of the reference markings relative to the markers is known, the depth of the medical device within the tissue may be determined by comparing the relative position of the reference markings and the markers to the starting point.

As used herein, the term “or” refers to an inclusive definition, for example, to mean “and/or” unless its context of usage clearly dictates otherwise. The term “and/or” refers to one or all of the listed elements or a combination of at least two of the listed elements.

As used herein, the phrases “at least one of” and “one or more of” followed by a list of elements refers to one or more of any of the elements listed or any combination of one or more of the elements listed.

As used herein, the terms “coupled” or “connected” refer to at least two elements being attached to each other either directly or indirectly. An indirect coupling may include one or more other elements between the at least two elements being attached. Either term may be modified by “operatively” and “operably,” which may be used interchangeably, to describe that the coupling or connection is configured to allow the components to interact to carry out described or otherwise known functionality. For example, a controller may be operably coupled to a resistive heating element to allow the controller to provide an electrical current to the heating element.

As used herein, any term related to position or orientation, such as “proximal,” “distal,” “end,” “outer,” “inner,” and the like, refers to a relative position and does not limit the absolute orientation of an embodiment unless its context of usage clearly dictates otherwise.

All scientific and technical terms used herein have meanings commonly used in the art unless otherwise specified. The definitions provided herein are to facilitate understanding of certain terms used frequently herein and are not meant to limit the scope of the present disclosure.

Reference will now be made to the drawings, which depict one or more aspects described in this disclosure. However, it will be understood that other aspects not depicted in the drawings fall within the scope of this disclosure. Like numbers used in the figures refer to like components, steps, and the like. However, it will be understood that the use of a reference character to refer to an element in a given figure is not intended to limit the element in another figure labeled with the same reference character. In addition, the use of different reference characters to refer to elements in different figures is not intended to indicate that the differently referenced elements cannot be the same or similar.

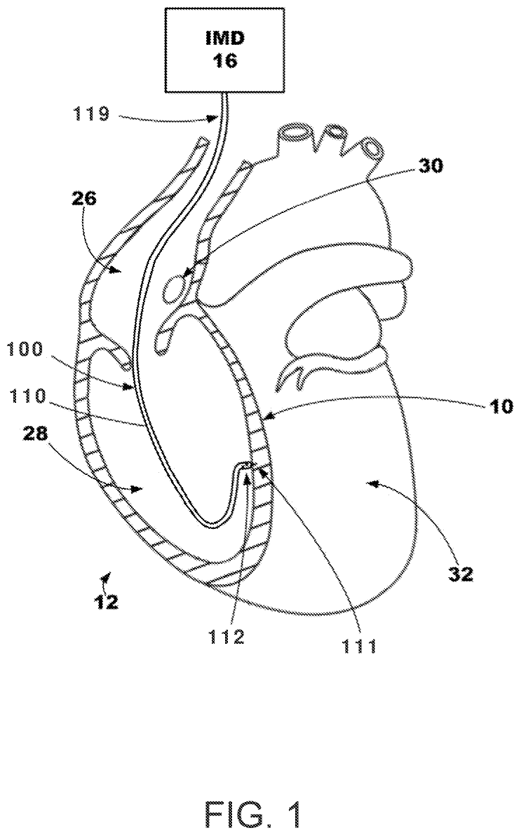

An illustrative implantable apparatus 100 (e.g., a pacing lead) implanted in a patient's heart 12 is illustrated in FIG. 1. More specifically, the implantable apparatus 100 may extend into the heart 12 of the patient to sense electrical activity of the heart 12 and/or to deliver electrical stimulation to the heart 12, and in particular, to sense electrical activity and/or to deliver electrical stimulation to the interventricular septum 10 of the heart 12. For example, the implantable apparatus 100 may be configured to deliver cardiac conduction system pacing therapy to the left and/or right bundle branches, the bundle of His, etc. from a location in the septum 10. Further, for example, the implantable apparatus 100 may be configured to deliver traditional myocardial pacing therapy to left and/or right ventricular myocardial tissue from a location in the septum 10. In the example shown in FIG. 1, the implantable apparatus 100 extends through one or more veins (not shown), the superior vena cava (not shown), the right atrium 26, and into the right ventricle 28. Then, the implantable apparatus 100 is positioned adjacent to the septum 10 in the right ventricle 28. Additionally, although a single lead is depicted in FIG. 1, it is to be understood that the implantable apparatus 100 may be used with one or more additional leads or leadless devices configured to sense electrical activity and/or deliver pacing therapy to the left ventricle, right ventricle, right atrium, etc. For example, the implantable apparatus 100 may be used in conjunction with a traditional left ventricular coronary sinus lead extending through one or more veins, the vena cava, the right atrium 26, and into the coronary sinus 30 to a region adjacent to the free wall of the left ventricle 32 of the heart 12 and/or a right atrial lead extending through one or more veins and the vena cava, and into the right atrium 26 of the heart 12.

Additionally, as diagrammatically shown, the implantable apparatus 100 may be operably coupled to an implantable medical device (IMD) 16. The IMD 16 may sense, among other things, electrical signals attendant to the depolarization and repolarization of the heart 12 via electrodes coupled to the implantable apparatus 100 or another lead such as a left ventricular lead, right atrial lead, etc. In some examples, the IMD 16 provides pacing therapy (e.g., pacing pulses) to the heart 12 based on the electrical signals sensed within the heart 12. The IMD 16 may be operable to adjust one or more parameters associated with the pacing therapy such as, e.g., A-V delay and other various timings, pulse wide, amplitude, voltage, burst length, etc. Further, the IMD 16 may be operable to use various electrode configurations to deliver pacing therapy, which may be unipolar, bipolar, quadripolar, or further multipolar. For example, a multipolar lead may include several electrodes that can be used for delivering pacing therapy. Hence, a multipolar lead system may provide, or offer, multiple electrical vectors to pace from. A pacing vector may include at least one cathode, which may be at least one electrode located on at least one lead, and at least one anode, which may be at least one electrode located on at least one lead (e.g., the same lead, or a different lead) and/or on the casing, or can, of the IMD. While improvement in cardiac function as a result of the pacing therapy may primarily depend on the cathode, the electrical parameters like impedance, pacing threshold voltage, current drain, longevity, etc. may be more dependent on the pacing vector, which includes both the cathode and the anode.

The implantable apparatus 100 may be described as including a body 110 extending from a proximal end region 119 to a distal end region 112. The proximal end region or portion 119 may be located proximate the IMD 16 for operably coupling thereto and the distal end region or portion 112 may be locatable, or positioned, at a target site, which in the example depicted in FIG. 1 is the interventricular septum 10.

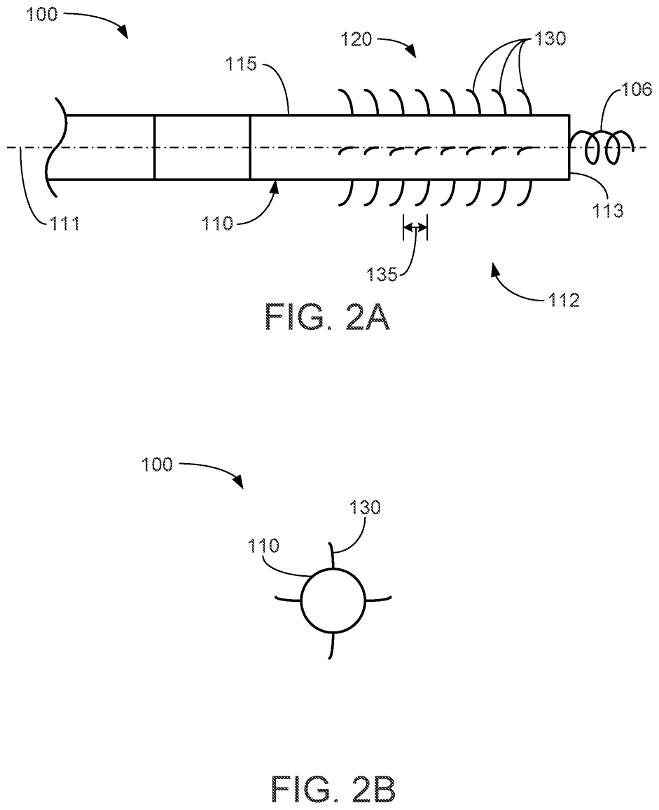

In the embodiment depicted, the distal end region 112 may extend along a distal end region axis 111 (e.g., as shown in FIG. 2A). More specifically, the distal end region axis 111 may be a straight, uncurving line, which the distal end region 112 may extend along, and thus, the distal end region 112 may also define a straight, uncurving line. In other embodiments, the distal end region 112 may not extend along an axis, and may, for example, define a variety of different shapes or curves, and the markers described further herein, may be configured to take into account the shape and curvature of the distal end region 112 to determine the penetration depth of the implantable apparatus 100.

FIGS. 2A-7 show various examples of markers 120 located on a distal end region 112 of an implantable apparatus 100 according to the present disclosure. The implantable apparatus 100 may include a body 110 defining the distal end region 112 extending along a distal end region axis 111. The body 110 (e.g., the distal end region 112) may be configured to be inserted into cardiac tissue of a patient's heart at a target site. For example, as described herein, the target site may include the interventricular septum 10 (e.g., as shown in FIG. 1).

In one or more embodiments, the implantable apparatus 100 may include a fixation element 106 extending from the distal end region 112 of the body 110 (e.g., at the distal end 113) and configured to couple (e.g., fixate or attach) the body 110 to the target site. In one embodiment, the fixation element 106 may be a helical fixation element that may be “screwed” into the tissue (e.g., the interventricular septum).

The implantable apparatus 100 may also include a plurality of markers 120 located along at least a portion of an outer surface 115 of the distal end region 112 of the body 110. The plurality of markers 120 may assist in determining the depth of the body 110 of the implantable apparatus 100 inserted into the tissue. For example, the plurality of markers 120 may be viewable under imaging to allow the user to determine the position of the body 110 of the implantable apparatus 100 relative to a surface of the tissue (e.g., through which the implantable apparatus 100 may be inserted).

The plurality of markers 120 may include any suitable material or coating such that the plurality of markers 120 may be viewable under imagining. For example, the plurality of markers 120 may be radiopaque, platinum-iridium structure, platinum-iridium alloy, aurum, tungsten loaded polymer, bismuth loaded polymer, barium loaded polymer, tantalum loaded polymer, etc. and viewable under imaging when located within the patient's heart. In one or more embodiments, the plurality of markers may include visible contrast or be configured to emit visible contrast such that the visible contrast may be viewable under imaging. The imaging contemplated herein may include any suitable process including, for example, X-ray, fluoroscopy, magnetic resonance imaging (MRI), computed tomography (CT), ultrasound imaging, echocardiography (e.g., of any kind), photoacoustic imaging, etc.

The plurality of markers 120 may define different configurations depending on whether the marker 120 is located within the tissue or outside of the tissue. For example, the plurality of markers 120 may initially define a configuration (e.g., appearance, shape, color, orientation, size, etc.) before the implantable apparatus 100 is inserted into the cardiac tissue. After the implantable apparatus 100 is inserted into the cardiac tissue, the markers 120 that are positioned within the cardiac tissue may define a different configuration. For example, the plurality of markers 120 may define a first configuration when located within the cardiac tissue and a second configuration when not located within the cardiac tissue (e.g., the first configuration being different than the second configuration). In other words, the body 110 of the implantable apparatus 100 may be configured to be inserted into the cardiac tissue such that only a portion (e.g., a first portion 122) of the plurality of markers 120 are located within the cardiac tissue. The remaining portion (e.g., a second portion 124) of the plurality of markers 120 are located outside of the cardiac tissue. Further, the first portion 122 of the plurality of markers 120 may define the first configuration and the second portion 124 of the plurality of markers 120 may define the second configuration.

Additionally, each of the plurality of markers 120 may be spaced apart along the distal end region axis 111 by a preset and/or fixed distance. Further, the orientation and positioning of the markers 120 relative to the body 110 may be set and known. For example, the plurality of markers 120 may be aligned with a feature of the body 110 (e.g., an anode location, an electrode location, a cathode location, a distal end 113, a center of gravity, a location of maximum tension/bending, etc.). Therefore, the markers 120 may be spaced apart by a known distance and positioned relative to the body 110 such that a user may be able to, e.g., count the number of markers 120 to determine a distance relative to the body 110. In other words, the user may be able to determine the depth of a body 110 inserted into tissue based on the markers 120. For example, the user may be able to identify which markers 120 are located within the cardiac tissue and, thereby, the depth of the body 110 of the implantable apparatus 100 located within the cardiac tissue.

Further, the plurality of markers 120 may be spaced apart in any suitable, known, way. For example, the plurality of markers 120 may be spaced apart in a preset manner such that the user knows the exact spacing between each marker of the plurality of markers 120. In one or more embodiments, each of the plurality of markers 120 may be evenly spaced apart along the distal end region axis 111. Also, each of the plurality of markers 120 may be spaced apart by any suitable distance (e.g., measured along the distal end region axis 111). Specifically, each of the plurality of markers 120 may be spaced apart by about 0.5 mm, 1 mm, 1.5 mm, 2 mm, etc.

The configurations of the plurality of markers 120 may be represented in a variety of different ways. For example, the configurations of the plurality of markers 120 may be represented by visual appearance, color (e.g., grayscale/density in image), shape, orientation, size, etc. As noted herein, the configuration of the plurality of markers 120 may change based on whether the marker 120 is within the tissue (e.g., in a first configuration) or not within the tissue (e.g., in a second configuration). As such, in one or more embodiments, the first configuration of the plurality of markers 120 may define a first visual appearance and the second configuration of the plurality of markers 120 may define a second visual appearance (e.g., that is different than the first visual appearance).

In one or more embodiments, the plurality of markers 120 may include filaments 130 extending from the outer surface 115 of the distal end region 112 of the body 110 as shown in FIGS. 2A and 2B. The filaments 130 may protrude from the outer surface 115 of the body 110 to a free end of the filament 130. In other words, each filament 130 may be coupled to the outer surface 115 of the body 110 at one end of the filament 130 and not coupled (e.g., free) at the other end of the filament 130.

The filaments 130 may be arranged in any suitable way. For example, the filaments 130 may be spaced apart along the distal end region axis 111 and around the axial direction of the body 110. For example, as shown in FIG. 2B, the filaments 130 may be positioned on four sides of the body 110 evenly spaced around the axial direction of the body 110. Further, as shown in FIG. 2A, the filaments 130 may extend in line with those four axial positions along the distal end region axis 111. In other embodiments, the filaments 130 may be positioned on one, two, three, five, six, seven, etc. sides of the body 110 around the axial direction.

Further, the filaments 130 may include any suitable number of filaments 130 positioned along the distal end region axis 111 and spaced apart in any suitable way. The number of filaments 130 and the spacing between each filament 130 may determine the total span or length of the filaments 130. For example, there may be about three to fifteen filaments 130 extending in each row along the distal end region axis 111. As shown in FIG. 2A, there are eight filaments arranged along the distal end region axis 111. Also, each filament 130 may be spaced apart by a distance 135 of about 0.5 mm to 3 mm (e.g., measured along the distal end region axis 111). As shown in FIG. 2A, each filament 130 is spaced apart by a distance 135 of about 1 mm. The filaments 130 may be equally spaced apart (e.g., each adjacent filament 130 spaced apart by the same distance) or spaced apart variably. It is noted that while the filaments 130 may be spaced apart by any suitable distance, the exact distance therebetween is a known distance such that the user may determine a length corresponding to any relative location along the filaments 130.

Therefore, as described herein, the filaments 130 (and known distances therebetween) may be used to determine the depth of the body 110 inserted into the tissue 102. For example, in addition to the filaments 130 being spaced apart by a known distance, the filaments 130 may be positioned relative to various components of the body 110 of the implantable apparatus 100 (e.g., an electrode, an anode, a cathode, etc.) by a fixed and known amount. As such, the filaments 130 may be used to determine the depth of the various components of the body 110 based on the known relationship between the filaments 130 and the various components.

Further, the total span or length of the filaments 130 along the distal end region axis 111 may limit the depth of the body 110 that may be measured. For example, only depth or distances that can be identified based on the filaments 130 may be used. In other words, in order to determine a depth of the body 110, the filaments 130 may be positioned on either side of the tissue 102 when the implantable apparatus 100 is inserted into the tissue 102. The user can then determine the depth or distance based on which filaments align with the surface of the tissue 102 (e.g., a point along the filaments 130 at which adjacent filaments have a different configuration or appearance). Additionally, the precision of the determined depth of the body is based on the distance between filaments 130. In other words, the closer together the filaments 130, the more precise the determined depth, and the farther apart the filaments 130, the less precise the determined depth (however, e.g., identifying the individual filaments 130 may be easier).

The filaments 130 may include (e.g., be formed of) any suitable material. For example, the filaments 130 may include platinum micro wire, aurum micro wire, tungsten micro wire, tantalum micro wire, etc. Specifically, the filaments 130 may be described as a threadlike (e.g., hair-like), soft, and/or slippery material. Further, the filaments 130 may include a flexible material that is configured to move or bend. As such, the filaments 130 may be configurable into various directions and/or orientations (e.g., depending on any external forces being applied to the filaments). For example, the filaments 130 may change configurations depending on whether or not the filament 130 is located within the tissue (e.g., the tissue moving or applying pressure/force to the filaments 130).

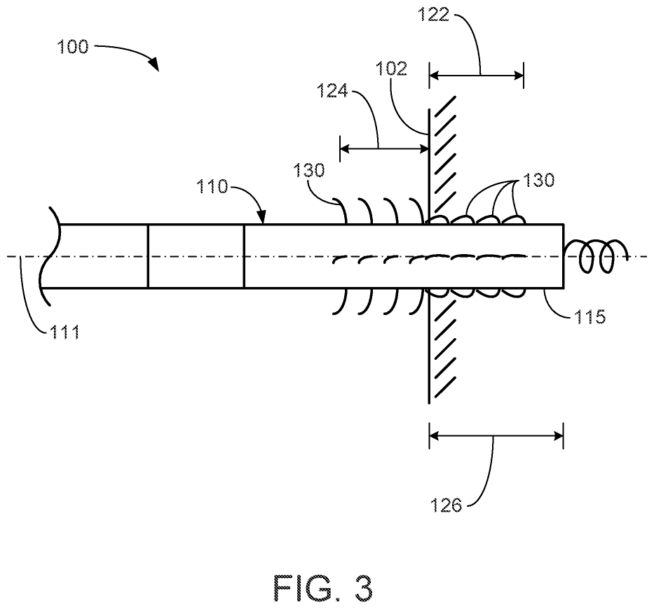

As shown in FIG. 3, a first portion 122 of the filaments 130 may be located within the tissue 102 and define a first configuration, and a second portion 124 of the filaments 130 may be located outside of the tissue 102 and define a second configuration. The first and second portions 122, 124 of filaments 130 are defined solely based on which filaments 130 are located inside of the tissue 102 and which filaments 130 are located outside of the tissue 102, respectively. Specifically, the first configuration of the filaments 130 (e.g., at the first portion 122) may define the filaments 130 as bending or pressed closer to the outer surface 115 of the body 110, while the second configuration of the filaments 130 (e.g., at the second portion 124) may define the filaments 130 unaffected and allowed to move unimpeded. Therefore, as the implantable apparatus 100 is moved into the tissue 102, the filaments 130 may be pressed against the outer surface 115 of the body 110 as the filaments 130 are inserted into the tissue 102 (with the implantable apparatus 100). In one or more embodiments, the filaments 130 that are pressed against the outer surface 115 of the body 110 (e.g., the first configuration) may revert back to being unaffected and unimpeded (e.g., the second configuration) when at least a portion of the body 110 is removed from the tissue 102 (e.g., the filaments 130 that correspond to the portion of the body 110 that is removed).

When viewed under imaging, a user may identify the first portion 122 of the filaments 130 and the second portion 124 of the filaments 130 based on the differing configuration or visual appearance of each portion of the filaments 130. For example, in one or more embodiments, the filaments 130 may be bent or broken when in the first configuration and continuous when in the second configuration. Also, in one or more embodiments, the filaments 130 may be configured to release visible contrast, when in the first configuration, that is viewable under imaging. In other words, when each separate filament 130 is inserted into the tissue with the body 110, the filament 130 may release visible contrast (e.g., due to bending or breaking of the filament 130) to assist in identifying the filaments that are within the tissue 102. Specifically, in one or more embodiments, the visible contrast may be contained within an interior volume of the filament 130 such that, when the filament 130 is inserted into the tissue 102, the filament 130 may break and release the visible contrast. In other embodiments, all of the filaments 130 may be coated with a visible contrast and a user may identify the shape and orientation of the filaments 130 to determine the tissue boundary.

Because the distance between each filament 130 is known and the position of the filaments 130 relative to the body 110 is known, the user may determine a length 126 of the body 110 within the tissue 102. For example, each filament 130 relates to a specific depth of the body 110 within the tissue 102. The user may identify the point along the filaments 130 at which adjacent filaments 130 define a different configuration, which indicates the location of the tissue boundary. For example, as shown in FIG. 3, the filament 130 outside of the tissue 102 (e.g., to the left of the tissue boundary) extends unimpeded and the filament 130 within the tissue 102 (e.g., to the right of the tissue boundary) is bent and forced towards the outer surface 115 of the body 110. This point of the filaments 130 that change visual appearance (e.g., configurations) between adjacent filaments 130 may then be used to determine the depth of the body 110 or, e.g., various components located on the body 110.

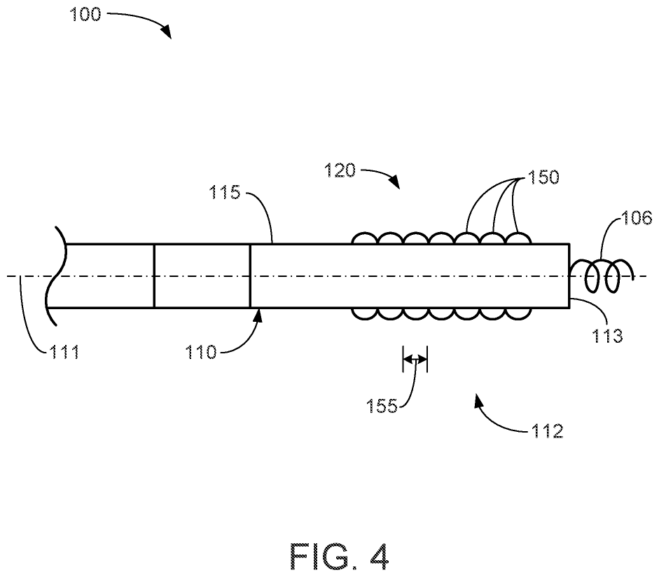

In one or more embodiments, the plurality of markers 120 may include inflatable balloons 150 extending from the outer surface 115 of the distal end region 112 of the body 110 as shown in FIG. 4. The balloons 150 may define an internal volume between the balloon 150 and the outer surface 115 of the body 110 that is inflatable (e.g., with a fluid). The balloons 150 may be inflatable such that a user can identify the balloons 150 under imaging to assist in determining a depth at which the body 110 is positioned within cardiac tissue.

The balloons 150 may be arranged in any suitable way. For example, the balloons 150 may be spaced apart along the distal end region axis 111 and around an axial direction of the body 110. Specifically, as shown in FIG. 4, the balloons 150 may be positioned on opposing sides of the body 110. In other embodiments, the balloons 150 may be positioned on one, three, four, etc. sides of the body 110 around the axial direction.

Further, the balloons 150 may include any suitable number of balloons positioned along the distal end region axis 111 and spaced apart in any suitable way. The number of balloons 150, the size of the balloons 150, and the spacing between each balloon 150 may determine the total span or length of the balloons 150. For example, there may be about three to fifteen balloons 150 extending in each row along the distal end region axis 111. As shown in FIG. 4, there are seven balloons arranged along the distal end region axis 111. Further, the balloons 150 may be positioned adjacent to one another along the distal end region axis 111 in any suitable way. For example, the balloons 150 may be positioned relative to one another such that there is minimal or no gap between adjacent balloons 150 along the distal end region axis 111. Additionally, each of the balloons 150 may define any suitable dimension (e.g., length 155) measured along the distal end region axis 111. For example, each balloon 150 may define a length 155 of about 0.5 mm to 3 mm, and, more specifically, a length 155 of about 1 mm.

The balloons 150 may include (e.g., be formed of) any suitable material. For example, the balloons 150 may include TPU, PeBax, Nylon, Latex, rubber, etc. In one or more embodiments, the balloons 150 may include a visible material that may be viewable under imaging. For example, the balloons 150 may include a radiopaque material, a platinum-iridium structure, platinum micro wire structure, aurum micro wire structure, tungsten micro wire structure, tantalum micro wire structure, powder, etc. Further, in one or more embodiments, the balloons 150 may include a visible contrast, within the balloons 150, that is visible under imaging. For example, the visible contrast may be a coating on the inside of the balloons 150 or may be a fluid that fills or inflates the balloons 150.

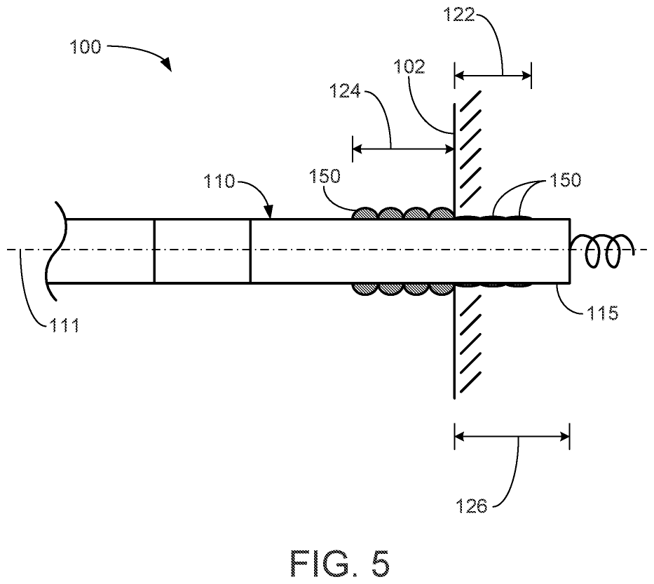

As shown in FIG. 4, the balloons 150 may be inflated when no external pressure or force is being applied to the balloons 150 (e.g., when within the heart, but not inserted into cardiac tissue). However, when the body 110 is inserted into tissue 102 and at least a portion of the balloons 150 are within the tissue 102, the balloons 150 may take on another configuration or appearance within the tissue 102. For example, as shown in FIG. 5, a first portion 122 of the balloons 150 may be located within the tissue 102 and define a first configuration, and a second portion 124 of the balloons 150 may be located outside of the tissue 102 and define a second configuration. The first configuration of the balloons 150 (e.g., at the first portion 122) may define the balloons as compressed or deflated, while the second configuration of the balloons 150 (e.g., at the second portion 124) may define the balloons 150 that are inflated. Therefore, as the body 110 of the implantable apparatus 100 is moved into the tissue 102, the corresponding balloons 150 at that portion of the body 110 may be compressed or deflated as the balloons 150 are inserted into the tissue 102 (e.g., the fluid from the balloons 150 within the tissue 102 moves into the body 110 or elsewhere). In one or more embodiments, if the body 110 is removed from the tissue 102, the deflated balloons 150 may reinflate once the pressure or force of the tissue 102 is no longer being applied to the balloons 150.

When viewed under imaging, a user may identify the first portion 122 of the balloons 150 and the second portion 124 of the balloons 150, and the point therebetween, based on whether the balloons 150 are inflated or deflated. In other words, the point at which adjacent balloons 150 are in different configurations correlates to a location of the tissue boundary. For example, as shown in FIG. 5, the balloon 150 outside of the tissue 102 (e.g., to the left of the tissue boundary) is inflated and the balloon 150 within the tissue 102 (e.g., to the right of the tissue boundary) is deflated. This point of the balloons 150 that change from inflated to deflated for adjacent balloons 150 may then be used to determine the depth of the body 110 or, e.g., various components located on the body 110. Specifically, because the width of each balloon 150, the distance between each balloon 150, and the position of the balloons 150 relative to the body 110 is known, a user may determine a length 126 of the body 110 within the tissue 102.

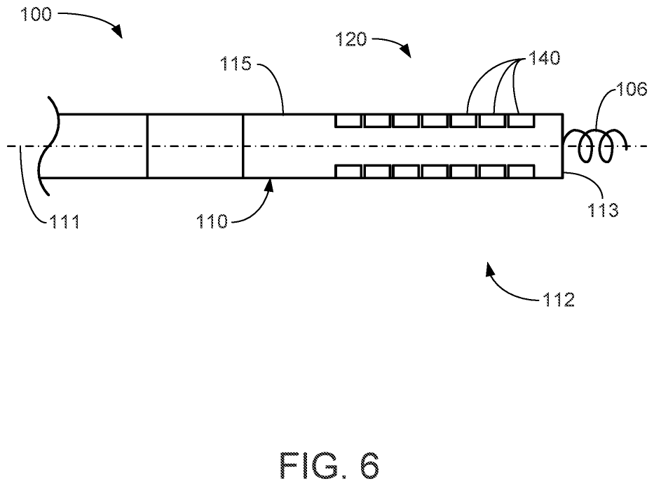

In one or more embodiments, the plurality of markers 120 may include a material coating 140 extending along the outer surface 115 of the distal end region 112 of the body 110 as shown in FIG. 6. The material coating 140 may assist in determining the depth of the body 110 inserted into the tissue 102. The material coating 140 may be arranged along the body 110 in any suitable way. For example, as shown in FIG. 6, the material coating 140 may define segmented portions spaced apart from one another. Also, as shown in FIG. 6, the material coating 140 may be located on opposing sides of the outer surface 115 of the body 110. In one or more embodiments, the material coating 140 define a continuous section extending along the distal end region 112 of the body 110 (e.g., around the entire circumference of the body 110 or only a portion or segments thereof).

The material coating 140 may interact with the tissue 102 in a variety of different ways to indicate which portion of the body 110 is located within the tissue 102. For example, in one or more embodiments, the material coating 140 may be pressure sensitive such that when a portion of the body 110 covered with the material coating 140 is located within the tissue 102, the material coating 140 changes the configuration or visual appearance (e.g., due to a pressure or force applied by the tissue 102). In other words, the visual appearance of the material coating 140 when outside of the tissue 102 is different than the visual appearance of the material coating 140 when within the tissue 102. The material coating 140 having pressure sensitive properties may include (e.g., be formed of) flexible coil, flexible stent, etc.

Also, in one or more embodiments, the material coating 140 may chemically interact with the tissue 102 such that when a portion of the body 110 covered with the material coating 140 is located within the tissue 102, the material coating 140 changes the configuration or visual appearance. In other words, the visual appearance of the material coating 140 when outside of the tissue 102 is different than the visual appearance of the material coating 140 when within the tissue 102. The material coating 140 having chemical interactive properties may include (e.g., be formed of) metal hollow microspheres, metal combined polymer hollow microspheres, metal nanospheres, metal combined polymer nanospheres, metal hollow nanospheres, metal combined polymer hollow nanospheres, etc.

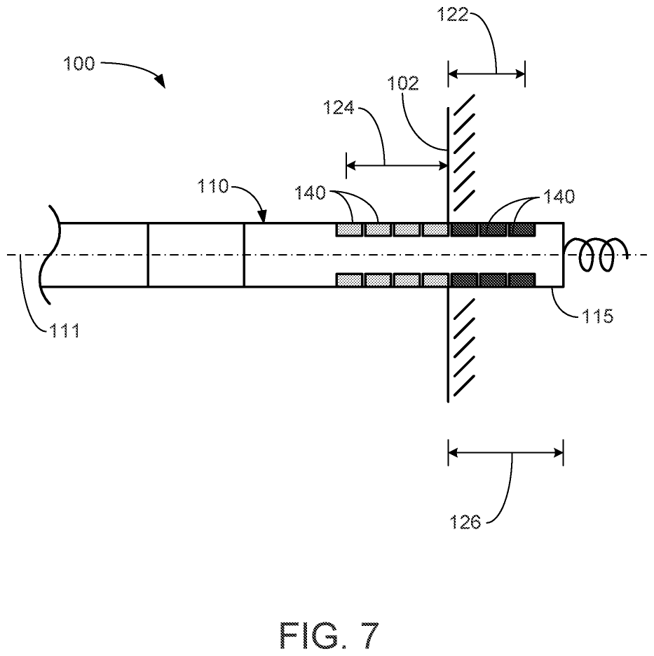

As shown in FIG. 7, a first portion 122 of the material coating 140 may be located within the tissue 102 and define a first visual appearance or first configuration (e.g., due to pressure/force, due to chemical interaction, etc.), and a second portion 124 of the material coating 140 may be located outside of the tissue 102 and define a second visual appearance or second configuration. Specifically, the visual appearance may include a variety of different suitable things. For example, the visual appearance may be related to shape, color (e.g., grayscale/density in image), patterns, size, etc.

As the body 110 of the implantable apparatus 100 is moved into the tissue 102, the corresponding material coating 140 at that portion of the body 110 may change configuration or visual appearance as inserted into the tissue 102. In one or more embodiments, if the body 110 is removed from the tissue 102, the material coating 140 may revert back to the original configuration or visual appearance once no longer within the tissue 102.

When viewed under imaging, a user may identify the first portion 122 of the material coating 140 and the second portion 124 of the material coating, and the point therebetween, based on the visual appearance or configuration of the material coating 140. In other words, the first visual appearance of the material coating 140 (e.g., at the first portion 122) may be different than the second visual appearance of the material coating 140 (e.g., at the second portion 124) such that a user may identify the transition of visual appearance along the body 110. The transition of visual appearance of the material coating 140 along the body 110 may correlate to the tissue boundary. For example, as shown in FIG. 7, the material coating 140 outside of the tissue 102 (e.g., to the left of the tissue boundary) is a lighter color and the material coating 140 within the tissue (e.g., to the right of the tissue boundary) is a darker color. This point of visual appearance transition of the material coating 140 may be used to determine the depth of the body 110 or, e.g., various components located on the body 110. Specifically, because the dimensions of the material coating 140 and the position of the material coating relative to the body 110 is known, a user may determine a length 126 of the body within the tissue 102.

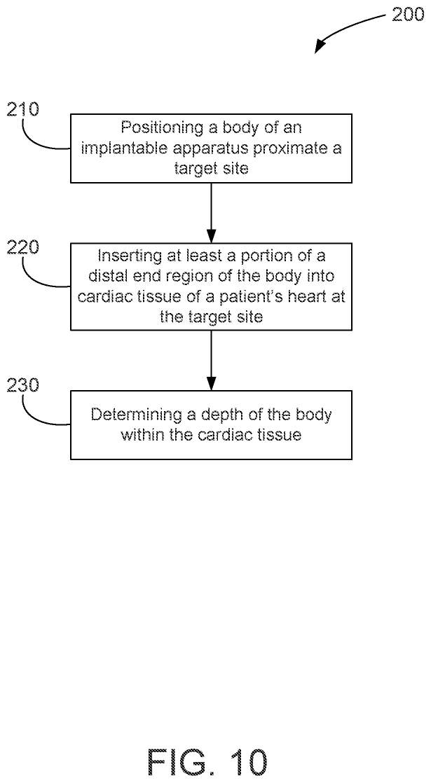

A flow diagram of a method 200 of determining a depth of an implantable apparatus in accordance with the present disclosure is illustrated in FIG. 10. The method 200 may include positioning 210 a body of an implantable apparatus proximate a target site (e.g., the interventricular septum) including cardiac tissue of a patient's heart. The implantable apparatus may include components and features as described in connection with FIGS. 2-7. The body of the implantable apparatus may define a distal end region extending along a distal end region axis. The implantable apparatus may also include a plurality of markers (e.g., filaments, balloons, material coating, etc.) located along at least a portion of an outer surface of the distal end region of the body.

The method 200 may also include inserting 220 at least a portion of the distal end region of the body into the cardiac tissue of the patient's heart at the target site such that a first portion of the plurality of markers are located within the cardiac tissue and a second portion of the plurality of markers are located outside of the cardiac tissue. The first portion of the plurality of markers may be configured differently than the second portion of the plurality of markers when the first portion is within the cardiac tissue. For example, the first portion may define a first configuration or first visual appearance and the second portion may define a second configuration or second visual appearance.

Further, the method 200 may include determining 230 a depth of the body within the cardiac tissue based on a length of the first portion of the plurality of markers. For example, the length of the first portion of the plurality of markers may correspond to a length of the body that is inserted into the tissue.

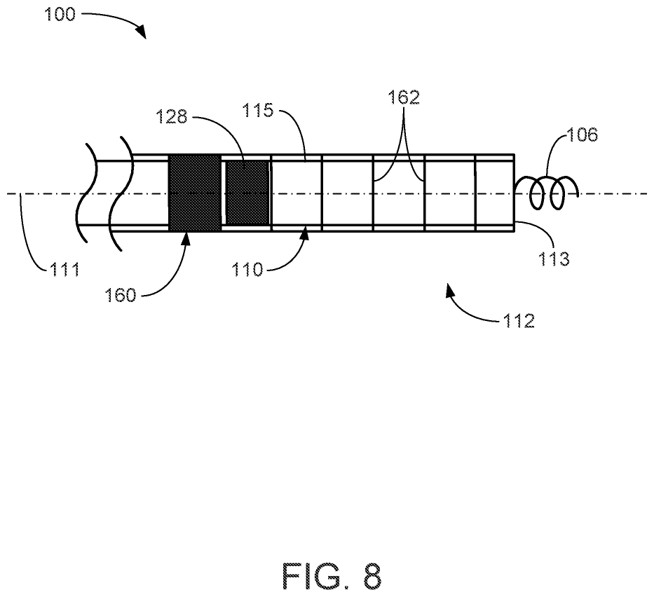

Another example of an illustrative implantable apparatus 100 including features to determine a depth of a body 110 of the implantable apparatus 100 inserted into cardiac tissue is illustrated in FIG. 8. The body 110 of the implantable apparatus 100 may define a distal end region 112 extending along a distal end region axis 111. The body 110 may be configured to be inserted into the cardiac tissue of a patient's heart at a target site (e.g., the interventricular septum). The implantable apparatus 100 may also include one or more markers 128 located along at least a portion of an outer surface 115 of the distal end region 112 of the body 110.

The implantable apparatus 100 may also include a jacket 160 configured to move relative to the body 110. The jacket 160 may be restricted from being inserted into the cardiac tissue such that the jacket 160 moves relative to the body 110 when the body 110 is inserted into the cardiac tissue. The jacket 160 may include reference markings 162 configured to be compared to the one or more markers 128 of the body 110 to determine the depth of the body 110 within the cardiac tissue. In other words, an initial positioning between the reference markings 162 and the one or more markers 128 may be compared to the positioning after the body 110 is inserted into the tissue to determine the depth of penetration.

Specifically, the one or more markers 128 and the reference markings 162 may be visible under imaging to allow a user to identify the relationship between the markers 128 of the body 110 and the reference markings 162 of the jacket 160. The one or more markers 128 and the reference markings 162 may include (e.g., be formed of) any suitable material that is viewable under imaging such as, e.g., radiopaque material, platinum-iridium structure, platinum-iridium alloy, aurum, tungsten loaded polymer, bismuth loaded polymer, barium loaded polymer, tantalum loaded polymer, etc. Also, in one or more embodiments, the jacket 160 may include a transparent material or a material through which the one or more markers 128 of the body 110 may be visible therethrough.

The reference markings 162 may be spaced apart in any suitable way. For example, the reference markings 162 may be spaced apart by about 0.5 mm to 3 mm. Specifically, the reference markings 162 may be spaced apart by about 1 mm.

The one or more markers 128 may be aligned in a known way with any component of the body 110 of the implantable apparatus 100 such that the depth of the component within the tissue may be determined. For example, the one or more markers 128 may be aligned with an anode or cathode location of the body 110. As such, the depth of the anode or cathode location of the body 110 may be determined based on the displacement of the jacket 160 relative to the body 110 (e.g., by comparing the one or more markers 128 with the reference markings 162). In one or more embodiments, the one or more markers 128 may be aligned with a distal end 113 of the body 110. As such, the depth of the distal end 113 of the body 110 may be determined based on the displacement of the jacket 160 relative to the body 110.

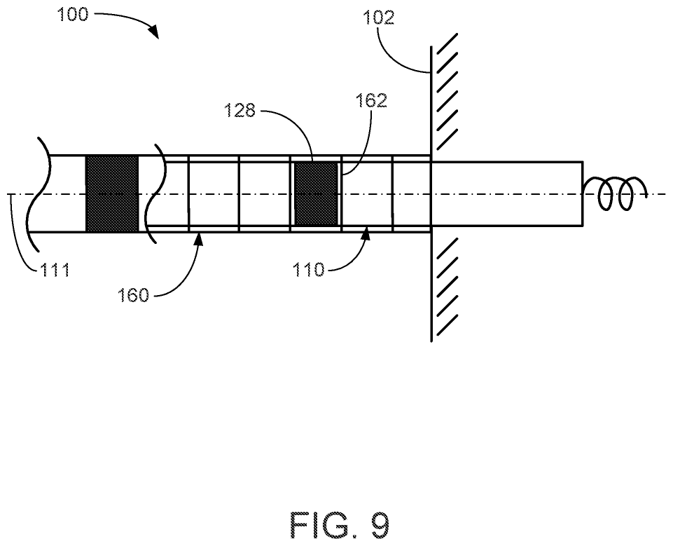

As shown in FIG. 9, the body 110 is inserted into the tissue 102 and the jacket 160 is restricted from entering the tissue 102. Therefore, the initial position of the reference markings 162 relative to the one or more markers 128 has shifted to a new position. Based on this difference, a user may determine a depth of the body 110 (or components thereof) within the tissue 102.

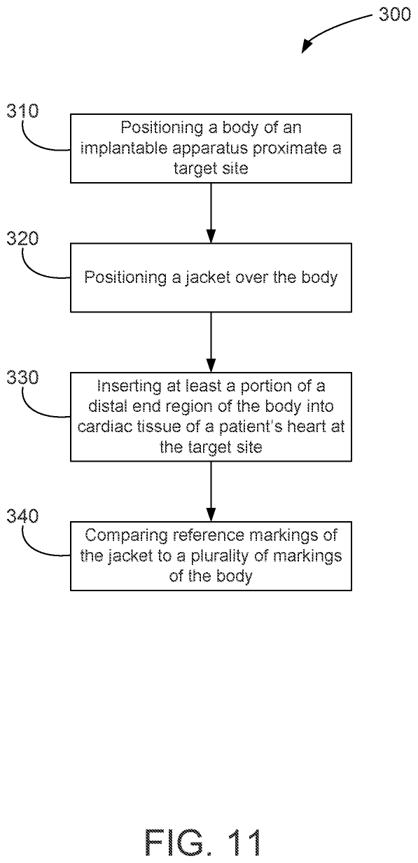

A flow diagram of a method 300 of determining a depth of a body of an implantable apparatus (e.g., the implantable apparatus as described in connection with FIGS. 8 and 9) within cardiac tissue is illustrated in FIG. 11. The method 300 may include positioning 310 a body of an implantable apparatus proximate a target site (e.g., the interventricular septum) including cardiac tissue of a patient's heart. The body may define a distal end region extending along a distal end region axis. The implantable apparatus may also include one or more markers located along at least a portion of an outer surface of the distal end region of the body.

The method 300 may also include positioning 320 a jacket over the body and inserting 330 at least a portion of the distal end region of the body into the cardiac tissue of the patient's heart at the target site. The jacket may include reference markings. The jacket may be restricted from being inserted into the cardiac tissue such that the jacket moves relative to the body. Further, the method 300 may include comparing 340 the reference markings of the jacket to the one or more markings of the body to determine a depth of the body within the cardiac tissue.

Unless otherwise indicated, all numbers expressing feature sizes, amounts, and physical properties used in the specification and claims may be understood as being modified either by the term “exactly” or “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein or, for example, within typical ranges of experimental error.

As used herein, the term “configured to” may be used interchangeably with the terms “adapted to” or “structured to” unless the content of this disclosure clearly dictates otherwise.

The singular forms “a,” “an,” and “the” encompass embodiments having plural referents unless its context clearly dictates otherwise.

As used herein, “have,” “having,” “include,” “including,” “comprise,” “comprising” or the like are used in their open-ended sense, and generally mean “including, but not limited to.” It will be understood that “consisting essentially of,” “consisting of,” and the like are subsumed in “comprising,” and the like.

Reference to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout are not necessarily referring to the same embodiment of the disclosure. Furthermore, the particular features, configurations, compositions, or characteristics may be combined in any suitable manner in one or more embodiments.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

### ILLUSTRATIVE EMBODIMENTS

While the present disclosure is not so limited, an appreciation of various aspects of the disclosure will be gained through a discussion of the specific examples and illustrative embodiments provided below. Various modifications of the examples and illustrative embodiments, as well as additional embodiments of the disclosure, will become apparent herein.

Thus, various embodiments described herein are disclosed. It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

A1. An implantable apparatus comprising:
a body defining a distal end region extending along a distal end region axis, wherein the body is configured to be inserted into cardiac tissue of a patient's heart at a target site; and
a plurality of markers located along at least a portion of an outer surface of the distal end region of the body, wherein the plurality of markers define a first configuration when located within the cardiac tissue and a second configuration when not located within the cardiac tissue, wherein the first configuration is different than the second configuration.
   A2. The implantable apparatus according to any A embodiment, wherein the plurality of markers are radiopaque and viewable under imaging when located within the patient's heart.
   A3. The implantable apparatus according to any A embodiment, wherein the body is configured to be inserted into the cardiac tissue such that only a portion of the plurality of markers are located within the cardiac tissue, wherein the portion of the plurality of markers define the first configuration and a remainder of the plurality of markers define the second configuration.
   A4. The implantable apparatus according to any A embodiment, wherein the plurality of markers comprise visible contrast when viewed under imaging and when in the first configuration.
   A5. The implantable apparatus according to any A embodiment, wherein the first configuration of the plurality of markers defines a first visual appearance under imaging and the second configuration of the plurality of markers defines a second visual appearance under imaging, wherein the first and second visual appearances are different.
   A6. The implantable apparatus according to any A embodiment, wherein the plurality of markers are viewed under imaging using fluoroscopy, magnetic resonance imaging (MRI), computed tomography (CT), ultrasound imaging, echocardiography, or photoacoustic imaging.
   A7. The implantable apparatus according to any A embodiment, wherein each of the plurality of markers are spaced apart along the distal end region axis by a preset distance.
   A8. The implantable apparatus according to embodiment A7, wherein each of the plurality of markers is evenly spaced apart along the distal end region axis.
   A9. The implantable apparatus according to any A embodiment, wherein the target site comprises the interventricular septum.
   A10. The implantable apparatus according to any A embodiment, wherein the plurality of markers comprise filaments extending from the outer surface of the distal end region of the body.
   A11. The implantable apparatus according to embodiment A10, wherein the filaments are bent or broken when in the first configuration.
   A12. The implantable apparatus according to embodiment A10, wherein the filaments are configured to release visible contrast, when in the first configuration, when viewed under imaging.
   A13. The implantable apparatus according to embodiment A10, wherein the filaments comprise a flexible material.
   A14. The implantable apparatus according to any A embodiment, wherein the plurality of markers comprise a material coating, wherein the plurality of markers define a first visual appearance when in the first configuration due to a pressure applied to the plurality of markers by the cardiac tissue, wherein the plurality of markers define a second visual appearance when in the second configuration, wherein the first and second visual appearances are different.
   A15. The implantable apparatus according to any A embodiment, wherein the plurality of markers comprise a material coating, wherein the plurality of markers define a first visual appearance when in the first configuration due to an interaction between the cardiac tissue and the material coating, wherein the plurality of markers define a second visual appearance when in the second configuration, wherein the first and second visual appearances are different.
   A16. The implantable apparatus according to any A embodiment, wherein the plurality of markers comprise inflatable balloons extending from the outer surface of the distal end region of the body.
   A17. The implantable apparatus according to embodiment A16, wherein the balloons are inflated when in the second configuration and compressed when in the first configuration.
   A18. The implantable apparatus according to embodiment A16, wherein the balloons comprise visible contrast, within the balloons, that is viewable under imaging.
   A19. The implantable apparatus according to embodiment A16, wherein the balloons comprise a visible material that is viewable under imaging.
   A20. The implantable apparatus according to any A embodiment, wherein the body comprises a pacing lead.
   A21. The implantable apparatus according to any A embodiment, further comprising a fixation element extending from the distal end region of the body and configured to couple the body to the target site.
   B1. A method comprising:
positioning a body of an implantable apparatus proximate a target site comprising cardiac tissue of a patient's heart, wherein the body defines a distal end region extending along a distal end region axis, wherein the implantable apparatus further comprises a plurality of markers located along at least a portion of an outer surface of the distal end region of the body;
inserting at least a portion of the distal end region of the body into the cardiac tissue of the patient's heart at the target site such that a first portion of the plurality of markers are located within the cardiac tissue and a second portion of the plurality of markers are located outside of the cardiac tissue, wherein the first portion of the plurality of markers is configured differently than the second portion of the plurality of markers when the first portion is within the cardiac tissue; and
determining a depth of the body within the cardiac tissue based on a length of the first portion of the plurality of markers.
   B2. The method according to any B embodiment, wherein the plurality of markers are radiopaque and viewable under imaging when located within the patient's heart.
   B3. The method according to any B embodiment, wherein the first portion of the plurality of markers is configured differently than the second portion of the plurality of markers based on visual appearance.
   B4. The method according to any B embodiment, wherein each of the plurality of markers are spaced apart along the distal end region axis by a preset distance.
   B5. The method according to embodiment B4, wherein each of the plurality of markers is spaced apart evenly along the distal end region axis.
   B6. The method according to any B embodiment, wherein the target site comprises the interventricular septum.
   B7. The method of according to any B embodiment, wherein the plurality of markers comprise filaments extending from the outer surface of the distal end region of the body.
   B8. The method according to embodiment B7, wherein the filaments located at the first portion of the plurality of markers are bent or broken.
   B9. The method according to embodiment B7, wherein the filaments located at the first portion of the plurality of markers are configured to release visible contrast when viewed under imaging.
   B10. The method according to any B embodiment, wherein the plurality of markers comprise a material coating, wherein the plurality of markers define a first visual appearance at the first portion due to a pressure applied to the plurality of markers by the cardiac tissue, wherein the plurality of markers define a second visual appearance at the second portion, wherein the first and second visual appearances are different.
   B11. The method according to any B embodiment, wherein the plurality of markers comprise a material coating, wherein the plurality of markers define a first visual appearance at the first portion due to an interaction between the cardiac tissue and the material coating, wherein the plurality of markers define a second visual appearance at the second portion, wherein the first and second visual appearances are different.
   B12. The method according to any B embodiment, wherein the plurality of markers comprise inflatable balloons extending from the outer surface of the distal end region of the body.
   B13. The method according to embodiment B12, wherein the balloons located at the second portion of the plurality of markers are inflated and the balloons located at the first portion of the plurality of markers are compressed.
   B14. The method according to embodiment B12, wherein the balloons comprise visible contrast, within the balloons, that is viewable under imaging.
   B15. The method according to embodiment B12, wherein the balloons comprise a visible material that is viewable under imaging.
   B16. The method according to any B embodiment, wherein the body comprises a pacing lead.
   B17. The method according to any B embodiment, further comprising attaching the body to the target site via a fixation element extending from the distal end region of the body.
   C1. An implantable apparatus comprising:
a body defining a distal end region extending along a distal end region axis, wherein the body is configured to be inserted into cardiac tissue of a patient's heart at a target site;
one or more markers located along at least a portion of an outer surface of the distal end region of the body; and
a jacket configured to move relative to the body, wherein the jacket is restricted from being inserted into the cardiac tissue such that the jacket moves relative to the body when the body is inserted into the cardiac tissue, wherein the jacket comprises reference markings, wherein the reference markings of the jacket are configured to be compared to the one or more markers of the body to determine a depth of the body within the cardiac tissue.
   C2. The implantable apparatus according to any C embodiment, wherein the jacket comprises a transparent material.
   C3. The implantable apparatus according to any C embodiment, wherein the one or more markers of the body are aligned with an anode location of the body.
   C4. The implantable apparatus according to any C embodiment, wherein the one or more markers of the body are aligned with a distal end of the body.
   C5. The implantable apparatus according to any C embodiment, wherein the reference markings are radiopaque and viewable under imaging when located within the patient's heart.
   C6. The implantable apparatus according to any C embodiment, wherein the one or more markers are radiopaque and viewable under imaging when located within the patient's heart.
   C7. The implantable apparatus according to any C embodiment, wherein the reference markings are viewed under imaging using fluoroscopy, magnetic resonance imaging (MRI), computed tomography (CT), ultrasound imaging, echocardiography, or photoacoustic imaging.
   C8. The implantable apparatus according to any C embodiment, wherein each of the reference markings are spaced apart by a preset distance.
   C9. The implantable apparatus according to embodiment C8, wherein each of the reference markings is evenly spaced apart along the distal end region.
   C10. The implantable apparatus according to any C embodiment, wherein the target site comprises the interventricular septum.
   C11. The implantable apparatus according to any C embodiment, wherein the body comprises a pacing lead.
   C12. The implantable apparatus according to any C embodiment, further comprising a fixation element extending from the distal end region of the body and configured to couple the body to the target site.
   D1. A method comprising:
positioning a body of an implantable apparatus proximate a target site comprising cardiac tissue of a patient's heart, wherein the body defines a distal end region extending along a distal end region axis, wherein the implantable apparatus further comprises one or more markers located along at least a portion of an outer surface of the distal end region of the body;
positioning a jacket over the body, wherein the jacket comprises reference markings;
inserting at least a portion of the distal end region of the body into the cardiac tissue of the patient's heart at the target site, wherein the jacket is restricted from being inserted into the cardiac tissue such that the jacket moves relative to the body; and
comparing the reference markings of the jacket to the one or more markers of the body to determine a depth of the body within the cardiac tissue.
   D2. The method according to any D embodiment, wherein the jacket comprises a transparent material.
   D3. The method according to any D embodiment, wherein the one or more markers of the body are aligned with an anode location of the body.
   D4. The method according to any D embodiment, wherein the one or more markers of the body are aligned with a distal end of the body.
   D5. The method according to any D embodiment, wherein the reference markings are radiopaque and viewable under imaging when located within the patient's heart.
   D6. The method according to any D embodiment, wherein the one or more markers are radiopaque and viewable under imaging when located within the patient's heart.
   D7. The method according to any D embodiment, wherein the reference markings are viewed under imaging using fluoroscopy, magnetic resonance imaging (MRI), computed tomography (CT), ultrasound imaging, echocardiography, or photoacoustic imaging.
   D8. The method according to any D embodiment, wherein each of the reference markings are spaced apart by a preset distance.
   D9. The method according to embodiment D8, wherein each of the reference markings is evenly spaced apart along the distal end region.
   D10. The method according to any D embodiment, wherein the target site comprises the interventricular septum.
   D11. The method according to any D embodiment, wherein the body comprises a pacing lead.
   D12. The method according to any D embodiment, further comprising attaching the body to the target site via a fixation element extending from the distal end region of the body.

1. An implantable apparatus comprising:
a body defining a distal end region extending along a distal end region axis, wherein the body is configured to be inserted into cardiac tissue of a patient's heart at a target site; and a plurality of markers located along at least a portion of an outer surface of the distal end region of the body, wherein the plurality of markers define a first configuration when located within the cardiac tissue and a second configuration when not located within the cardiac tissue, wherein the first configuration is different than the second configuration.

2. The implantable apparatus of claim 1, wherein the plurality of markers are radiopaque and viewable under imaging when located within the patient's heart.

3. The implantable apparatus of claim 1, wherein the body is configured to be inserted into the cardiac tissue such that only a portion of the plurality of markers are located within the cardiac tissue, wherein the portion of the plurality of markers define the first configuration and a remainder of the plurality of markers define the second configuration.

4. The implantable apparatus of claim 1, wherein the first configuration of the plurality of markers defines a first visual appearance under imaging and the second configuration of the plurality of markers defines a second visual appearance under imaging, wherein the first and second visual appearances are different.

5. The implantable apparatus of claim 1, wherein each of the plurality of markers are spaced apart along the distal end region axis by a preset distance.

6. The implantable apparatus of claim 1, wherein the target site comprises the interventricular septum.

7. The implantable apparatus of claim 1, wherein the plurality of markers comprise filaments extending from the outer surface of the distal end region of the body.

8. The implantable apparatus of claim 7, wherein the filaments are bent or broken when in the first configuration.

9. The implantable apparatus of claim 1, wherein the plurality of markers comprise a material coating, wherein the plurality of markers define a first visual appearance when in the first configuration due to a pressure applied to the plurality of markers by the cardiac tissue, wherein the plurality of markers define a second visual appearance when in the second configuration, wherein the first and second visual appearances are different.

10. The implantable apparatus of claim 1, wherein the plurality of markers comprise a material coating, wherein the plurality of markers define a first visual appearance when in the first configuration due to an interaction between the cardiac tissue and the material coating, wherein the plurality of markers define a second visual appearance when in the second configuration, wherein the first and second visual appearances are different.

11. The implantable apparatus of claim 1, wherein the plurality of markers comprise inflatable balloons extending from the outer surface of the distal end region of the body.

12. The implantable apparatus of claim 11, wherein the balloons are inflated when in the second configuration and compressed when in the first configuration.

13. The implantable apparatus of claim 11, wherein the balloons comprise a visible material that is viewable under imaging.

14. The implantable apparatus of claim 1, wherein the body comprises a pacing lead.

15. The implantable apparatus of claim 1, further comprising a fixation element extending from the distal end region of the body and configured to couple the body to the target site.

16. A method comprising:
positioning a body of an implantable apparatus proximate a target site comprising cardiac tissue of a patient's heart, wherein the body defines a distal end region extending along a distal end region axis, wherein the implantable apparatus further comprises a plurality of markers located along at least a portion of an outer surface of the distal end region of the body;
inserting at least a portion of the distal end region of the body into the cardiac tissue of the patient's heart at the target site such that a first portion of the plurality of markers are located within the cardiac tissue and a second portion of the plurality of markers are located outside of the cardiac tissue, wherein the first portion of the plurality of markers is configured differently than the second portion of the plurality of markers when the first portion is within the cardiac tissue; and
determining a depth of the body within the cardiac tissue based on a length of the first portion of the plurality of markers.

17. The method of claim 16, wherein the first portion of the plurality of markers is configured differently than the second portion of the plurality of markers based on visual appearance.

18. The method of claim 16, wherein each of the plurality of markers are spaced apart along the distal end region axis by a preset distance.

19. The method of claim 16, wherein the target site comprises the interventricular septum.

20. The method of claim 16, wherein the plurality of markers comprise filaments extending from the outer surface of the distal end region of the body.
